(19) United States
(12) Patent Application Publication
(10) Pub. No.: US 2006/0024823 A1
(43) Pub. Date: Feb. 2, 2006

(54) Cell culture carriers, method for manufacturing cell culture carriers and method for culturing cells

(75) Inventors: Tsuyoshi Ishikawa, Tokyo (JP); Ken Sugo, Saitama (JP)

Correspondence Address: GREENBLUM & BERNSTEIN, P.L.C., 1950 ROLAND CLARKE PLACE, RESTON, VA, 20191, US

(73) Assignee: PENTAX Corporation, Tokyo (JP)

(21) Appl. No.: 11/190,868

(22) Filed: Jul. 28, 2005

(30) Foreign Application Priority Data
Jul. 28, 2004 (JP) 2004-220870

(51) Int. Cl. C12M 1/00; C12N 5/00

(52) U.S. Cl. 435/325; 435/289.1

(57) ABSTRACT

Cell culture carriers that allow cells to efficiently adhere thereto and sufficiently grow thereon and from which the grown cells can be easily removed or detached, a method for manufacturing cell culture carriers that enables to easily and reliably manufacture such cell culture carriers and a method for culturing cells using such cell culture carriers are disclosed. The cell culture carrier includes a base material having a granular shape and a coating layer that is provided so as to cover the surface of the base material, the coating layer is mainly made of calcium phosphate-based apatite in which a part of calcium is deficient. Such cell culture carriers are utilized in cell culture in which cells adhere to and grow on the surface of cell culture carriers, particularly in three-dimensional high-density culture (suspension culture). The Ca deficiency rate in the calcium phosphate-based apatite in which a part of calcium is deficient is preferably in the range of 1 to 30 mol %.

### BACKGROUND OF THE INVENTION

### Field of the Invention

The present invention relates to cell culture carriers, a method for manufacturing cell culture carriers and a method for culturing cells (cell culturing method), and more specifically relates to cell culture carriers, a method for manufacturing the cell culture carriers and a method for culturing cells using the cell culture carriers.

In recent years, cell culture technology is used in various industrial and research fields such as cell tissue engineering, safety tests of drugs, production of proteins for treatment or diagnosis purposes, and the like.

Recently, in order to culture a large number of anchorage-dependent cells efficiently, cell culture is carried out by three-dimensional high-density culture (suspension culture) instead of plate culture which is commonly used. While the plate culture employs a culture flask, the suspension culture employs a number of carriers which serve as scaffolds on which cells are cultured.

In such three-dimensional high-density culture, various types of cell culture carriers that are made of, for example, polystyrene, diethylaminoethyl (DEAE) cellulose, polyacrylamide and the like are used.

However, depending on the kind of cell or culture condition, there is a case that cells hardly adhere to these carriers, or cells hardly grow on these carriers even if they have adhered thereto.

Therefore, cell culture carriers whose base materials are coated with hydroxyapatite have recently been proposed (see U.S. Patent Application Publication No. 2003-162287).

Since cells have the property of adhering to calcium, they can efficiently adhere to the cell culture carriers described above.

On the other hand, however, in this cell culture carriers, there is a problem in that when utilizing cells that have grown on the surfaces of the cell culture carriers, it is difficult to remove or detach the cells from the carriers. Further, there is also a problem in that the growing rate of cells is likely to decrease.

### SUMMARY OF THE INVENTION

It is therefore an object of the present invention to provide cell culture carriers that allow cells to efficiently adhere thereto and sufficiently grow thereon and from which the grown cells can be easily removed or detached, a method for easily and reliably manufacturing the cell culture carriers and a method for culturing cells using the cell culture carriers.

In order to achieve the above object, the present invention is directed to cell culture carriers in which each of the cell culture carriers has a surface to which cells are allowed to adhere so that the adhering cells grow on the surfaces thereof wherein each of the cell culture carriers has at least a surface thereof including its vicinities mainly made of calcium phosphate-based apatite in which a part of Ca is deficient.

According to the present invention described above, it is possible to obtain cell culture carriers that allow cells to efficiently adhere thereto and sufficiently grow thereon and from which the grown cells can be easily removed or detached.

In the cell culture carriers according to the present invention, it is preferred that the Ca deficiency rate of the calcium phosphate-based apatite in which a part of Ca is deficient is 1 to 30 mol %.

This allows cells to more efficiently adhere to the cell culture carriers and more sufficiently grow thereon. Further, the grown cells can be more easily removed or detached from the cell culture carriers.

In the cell culture carriers according to the present invention, it is also preferred that the density of each of the cell culture carriers is in the range of 1.01 to 1.5 g/cm³.

This makes it possible for the cell culture carriers to suspend in a culture solution more uniformly, and thereby allowing the cells to more efficiently adhere to the cell culture carriers.

In the cell culture carriers according to the present invention. It is also preferred that each of the cell culture carriers has a particle shape.

The cell culture carriers each having such a shape allow cells to equally adhere to each carrier and more efficiently grow on the surfaces of the cell culture carriers. Further, the cell culture carriers each having a granular shape can be suspended in a culture solution more uniformly. Therefore, the cell culture carriers have increased opportunities to make contact with cells, thereby enabling the cells to more efficiently adhere thereto.

In the cell culture carriers according to the present invention, it is also preferred that an average particle size of the cell culture carriers is in the rage of 10 to 2000 μm.

According to the cell culture carriers having such an average particle size, it is possible for cells to adhere to and grow on the surfaces thereof since the surface area of each cell culture carrier is sufficiently large with respect to the size of each cell.

In the cell culture carriers according to the present invention, it is also preferred that the calcium phosphate-based apatite in which a part of Ca is deficient is mainly made of hydroxyapatite in which a part of Ca is deficient.

Since hydroxyapatite is used as a biomaterial, cells can highly efficiently adhere thereto, and thus there is particularly less possibility of damaging the cells. For this reason, the Ca deficient hydroxyapatite can also have such properties, and therefore it is particularly suitably used as the calcium phosphate-based apatite in which a part of Ca is deficient.

In the cell culture carriers according to the present invention, it is also preferred that each of the cell culture carriers comprises a base material having a surface and a coating layer formed so as to coat the surface of the base material, the coating layer being mainly made of calcium phosphate-based apatite in which a part of Ca is deficient.

According to this structure, the shape, size and physical properties (density and the like) of the cell culture carriers can be preferably adjusted by appropriately setting the shape, size and physical properties of the base material.

In the cell culture carriers according to the present invention, it is also preferred that the base material is mainly made of a resin material.

According to this structure, it is possible to more easily adjust the shape, size and physical properties (specific gravity) of the cell culture carriers.

In the cell culture carriers according to the present invention, it is also preferred that the resin material is mainly composed of at least one of polyamide and epoxy resin.

By using such a base material mainly composed of at least one of polyamide and epoxy resin, the following effect can be obtained. Namely, since the density (specific gravity) of the above material is close to that of water, it is easy to adjust the density (specific gravity) of the cell culture carriers to a value close to that of water. Further, such cell culture carriers having a density close to that of water can be dispersed uniformly in a culture solution with mild agitation. Furthermore, the above material has high adhesiveness to Ca deficient apatite, thus making it possible to reliably coat the surface of the base material with the Ca deficient apatite. Morevoer, since the above material has excellent heat resistance, it is possible to obtain cell culture carriers having high heat resistance. Such cell culture carriers can be subjected to autoclave sterilization prior to cell culture.

In the cell culture carriers according to the present invention, it is also preferred that the base material contains a magnetic material.

According to this modification, the cell culture carriers can be moved in a culture solution when a magnetic field is applied thereto, thereby agitating the culture solution by themselves. As compared to an agitation using a stirring bar, the agitation of the culture solution by the cell culture carriers described above allows the culture solution to be agitated more uniformly and gently (mildly). Therefore, it is possible for the cells to easily adhere to the surfaces of the cell culture carriers, and it is also possible to prevent the cells from being removed or detached therefrom due to the collision between the cell culture carriers. Further, since nutrition is equally supplied to the cells, the cells grow more efficiently.

Another aspect of the present invention is directed to cell culture carriers in which each of the cell culture carriers has a surface to which cells are allowed to adhere so that the adhering cells grow on the surfaces thereof, wherein each of the cell culture carriers has at least a surface thereof including its vicinities mainly made of apatite in which a part of bivalent element thereof is deficient.

According to the invention of this aspect, it is possible to obtain cell culture carriers that allow cells to efficiently adhere thereto and sufficiently grow thereon and from which the grown cells can be easily removed or detached.

Other aspect of the present invention is directed to a method for manufacturing the cell culture carriers as described above. The manufacturing method comprises: a first step for obtaining slurry containing calcium phosphate-based apatite and phosphoric acid; a second step for drying the slurry to obtain powder; and a third step for sintering the powder to obtain sintered powder which is mainly formed of the calcium phosphate-based apatite in which a part of Ca is deficient by reacting the calcium phosphate-based apatite and the phosphoric acid in the powder with keeping the apatite structure to thereby produce the calcium phosphate-based apatite in which a part of Ca is deficient.

According to this manufacturing method, it is possible to easily and reliably manufacture cell culture carriers that allow cells to efficiently adhere thereto and sufficiently grow thereon and from which the grown cells can be easily removed or detached.

The other aspect of the present invention is directed to a method for manufacturing the cell culture carriers as defined in the second aspect of the present invention. The manufacturing method comprises: a first step for obtaining slurry containing calcium phosphate-based apatite and phosphoric acid; a second step for drying the slurry to obtain powder; a third step for sintering the powder to obtain sintered powder which is mainly formed of the calcium phosphate-based apatite in which a part of Ca is deficient by reacting the calcium phosphate-based apatite and the phosphoric acid in the powder with keeping the apatite structure to thereby produce the calcium phosphate-based apatite in which a part of Ca is deficienta; and a fourth step for coating a base material with the sintered powder.

According to this manufacturing method, it is possible to easily and reliably manufacture cell culture carriers that allow cells to efficiently adhere thereto and sufficiently grow thereon and from which the grown cells can be easily removed or detached. Further, the shape, size and physical properties (density and the like) of such cell culture carriers can be easily adjusted by appropriately setting the shape, size and physical properties of the base material.

In the manufacturing method described above, it is preferred that a mixing ratio of the calcium phosphate-based apatite and the phosphoric acid is in the range of 6:1 to 1:1 in a molar ratio in the first step.

According to this manufacturing method, it is possible to provide cell culture carriers that allow cells to more efficiently adhere thereto and more sufficiently grow thereon and from which the grown cells can be more easily removed or detached.

In the manufacturing method described above, it is also preferred that the sintering temperature in the third step is equal to or less than 1000° C.

According to this method, it is possible to produce the calcium phosphate-based apatite in which a part of Ca is deficient more reliably.

In the manufacturing method described above, it is also preferred that the sintering time in the third step is of 0.1 to 10 hours.

According to this method, it is also possible to produce the calcium phosphate-based apatite in which a part of Ca is deficient more reliably.

In the manufacturing method described above, it is preferred that the sintering in the third step is carried out in atmospheric air.

According to this, it is possible to efficiently produce the calcium phosphate-based apatite in which a part of Ca is deficient as well as to prevent the manufacturing cost from being increased.

Yet other aspect of the present invention is directed to a method for culturing cells, in which the cell culture is carried out by using the cell culture carriers as described above.

According to this cell culturing method, it is possible for the cells to efficiently grow on the surfaces of the cell culture carriers.

Yet other aspect of the present invention is directed to a method for culturing cells. In the cell culturing method, in a culture solution containing the cell culture carriers as described above and cells, the cells are brought into contact with the cell culture carriers so that the cells adhere to the surfaces of the cell culture carriers and grow thereon.

According to this cell culturing method, it is possible for the cells to efficiently grow on the surfaces of the cell culture carriers.

In this cell culturing method, the cells that have grown on the cell culture carriers can be easily removed or detached therefrom and thereby it is possible to utilize the cells for various purposes.

These and other objects, structures and results of the present invention will be apparent more clearly when the following detailed description of the preferred embodiments is considered taken in conjunction with the accompanying drawings.

### BRIEF DESCRIPTION OF THE DRAWINGS

FIG. 1 is a cross-sectional view of a first embodiment of a cell culture carrier according to the present invention.

FIG. 2 is a cross-sectional view of a second embodiment of the cell culture carrier according to the present invention.

FIG. 3 is a graph showing an x-ray diffraction pattern of sintered powder sintered at 700° C.

FIG. 4 is a graph showing an x-ray diffraction pattern of sintered powder sintered at 1100° C.

FIG. 5 is a graph showing an x-ray diffraction pattern of sintered powder sintered at 1200° C.

FIG. 6 is a schematic perspective view of a cell culture apparatus used in the present invention.

### DETAILED DESCRIPTION OF THE INVENTION

Hereinafter, cell culture carriers, a method for manufacturing cell culture carriers and a method for culturing cells (cell culturing method) according to the present invention will be described in detail based on preferred embodiments shown in the appended drawings.

### First Embodiment

First, the first embodiment of the cell culture carriers of the present invention will be described.

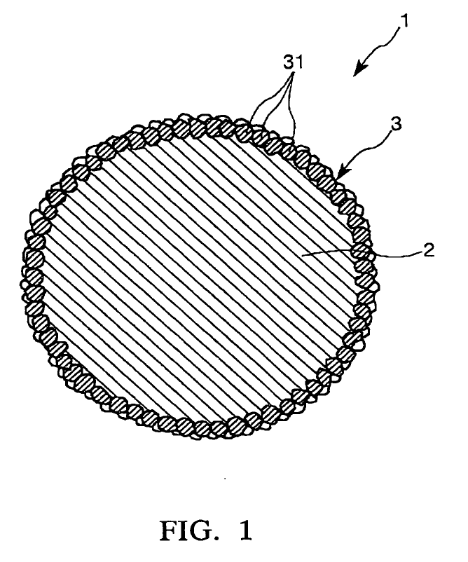

FIG. 1 is a cross-sectional view of a cell culture carrier according to the first embodiment of the present invention.

A cell culture carrier 1 shown in FIG. 1 comprises a base material 2 that is mainly made of a resin material and a coating layer 3 that is provided so as to cover the surface of the base material 2. The coating layer 3 is mainly made of calcium phosphate-based apatite in which a part of calcium is deficient (hereinafter, referred to as “Ca deficient apatite”).

Such a cell culture carrier 1 is utilized in cell culture in which cells are allowed to adhere to the surfaces of the cell culture carriers 1 and grow thereon, especially in three-dimensional high-density culture (suspension culture).

Examples of the three-dimensional high-density culture include microcarrier culture, spinner culture, rotary shake culture, rotation culture and the like. Among these methods, the cell culture carrier 1 is preferably used in the microcarrier culture. According to the microcarrier culture, it is possible to cultivate a large amount of cells with the extreme efficiency.

The cell culture carrier 1 is formed into a granular shape (substantially spherical granular shape) due to the base material 2 having a granular shape (substantially spherical shape in the structure shown in FIG. 1). The cell culture carrier 1 having such a shape allows cells to equally adhere to and more efficiently grow on the surfaces of the cell culture carriers 1. Further, the cell culture carriers 1 each having a granular shape can be suspended in a culture solution more uniformly. Therefore, the cell culture carriers 1 have increased opportunities to make contact with cells, thereby allowing the cells to more efficiently adhere thereto.

The size of the cell culture carrier 1 is not limited to any specific value. However, when the maximum length of a cell which is allowed to adhere to the cell culture carrier 1 is defined as L1 (μm), and the average particle size of the cell culture carriers 1 is defined as L2 (μm), L2/L1 is preferably in the range of about 2 to 100, and more preferably about 5 to 50.

Specifically, L2 is preferably in the range of about 10 to 2000 μm, more preferably in the range of about 50 to 1000 μm and even more preferably in the range of about 100 to 300 μm.

By setting L2/L1 to a value within the above range, it is possible to sufficiently increase the surface area of each cell culture carrier 1 with respect to the size of the cell, thereby allowing the cells to adhere to and grow on the surfaces of the cell culture carriers 1 more easily. If the average particle size of the cell culture carriers 1 is too small, there is a tendency that not only cells cannot efficiently adhere thereto, but also agglutination is likely to occur between cell culture carriers 1. On the other hand, if the average particle size of the cell culture carriers 1 is too large, the settling speed of the cell culture carriers 1 in a culture solution becomes higher, and thus the agitation speed has to be necessarily higher during cell culture. In such a case, the cell culture carriers 1 are likely to come into collision with each other. As a result, there is a possibility that the cells adhering to the surfaces of the cell culture carriers 1 are damaged.

Further, it is preferred that the density of the cell culture carrier 1 is close to that of water for enabling the cell culture carriers 1 to suspend in a culture solution more uniformly. Specifically, the density of the cell culture carrier 1 is preferably in the range of about 1.01 to 1.5 g/cm³, and more preferably in the range of about 1.02 to 1.2 g/cm³. By setting the density of the cell culture carrier 1 to a value within the above range, it is possible for the cell culture carriers 1 to suspend in a culture solution more uniformly, thereby enabling cells to adhere to the cell culture carriers 1 more efficiently.

The shape, size (average particle size or the like) and physical properties (density and the like) of such a cell culture carrier 1 can be adjusted by appropriately setting the shape, size, physical properties and the like of the base material 2.

For example, the density of the cell culture carrier 1 can be adjusted by appropriately setting the constituent material of the base material 2 and a form thereof (for example, porous, hollow structure and the like).

The base material 2 is mainly made of a resin material. The use of such a base material 2 makes it possible to more easily adjust the shape, size and properties (specific gravity, and the like) of the cell culture carrier 1.

In the case of manufacturing a cell culture carrier 1 having the above-mentioned average particle size, the average particle size of the base material 2 is preferably in the range of about 10 to 2000 μm, more preferably In the range of about 50 to 500 μm and even more preferably in the range of about 100 to 300 μm.

Further, in the case of manufacturing cell culture carriers 1 having the above-mentioned density, the density of the base material 2 is preferably in the range of about 1.01 to 1.5 g/cm³, and more preferably in the range of about 1.02 to 1.2 g/cm³.

As for the resin material used in the base material 2, various thermosetting resins and various thermoplastic resins can be used. Examples of the thermoplastic resins include polyamide, polyethylene, polypropylene, polystyrene, polyimide, an acrylic resin, and thermoplastic polyurethane. Further, examples of the thermosetting resins include an epoxy resin, a phenol resin, a melamine resin, a urea resin, unsaturated polyester, an alkyd resin, a thermosetting polyurethane, and ebonite. These resin materials may be used alone or in combination of two or more.

Among these resin materials, one that is mainly made of at least one of polyamide and an epoxy resin is particularly suitable. By using such a base material 2, the following effect can be obtained.

Since the density (specific gravity) of the above material is close to that of water, it is also easy to adjust the density (specific gravity) of the cell culture carrier 1 to a value close to that of water. Such cell culture carriers can be dispersed uniformly in a culture solution with mild agitation.

Further, the above material has high adhesiveness to Ca deficient apatite, thus making it possible to reliably coat the surface of the base material 2 with the Ca deficient apatite.

Furthermore, since the above material has excellent heat resistance, it is possible to obtain cell culture carriers 1 having high heat resistance. Such cell culture carriers 1 can be subjected to autoclave sterilization prior to cell culture.

Moreover, the resin material may be colored with organic pigment, inorganic pigment, acid dye, basic dye, or the like.

The coating layer 3 is provided so as to cover the substantially entire surface of the base material 2. As described above, the coating layer 3 is mainly formed of Ca deficient apatite (calcium phosphate-based apatite in which a part of calcium is deficient).

In this regard, it is to be noted that calcium phosphate-based apatite has a hexagonal crystal structure represented by a composition formula of Ca₁₀(PO₄)₆X₂ and the Ca deficient apatite is one in which the amount of Ca is reduced relative to calcium phosphate-based apatite represented by the above composition formula.

Therefore, the Ca deficient apatite includes one in which the relative amount of Ca is reduced due to addition of “PO₄” and “X” to calcium phosphate-based apatite represented by the above composition formula, and also includes one in which the relative amount of Ca is reduced due to elimination of Ca from calcium phosphate-based apatite represented by the above composition formula.

Further, in a culture solution, proteins which are involved in growth of cells are existing, and normally, such proteins adhere (are adsorbed) to the cell culture carriers 1 at first, and then cells adhere to the cell culture carriers 1 through these proteins. The protein has a negative electric charge and thus adheres to Ca having a positive electric charge. Therefore, various types of calcium phosphate-based compounds can adsorb the protein well, thereby enabling cells to efficiently adhere thereto.

Further, through the study by the inventor of the present invention, it has been found that among various calcium phosphate-based compounds, one having apatite structure (namely, calcium phosphate-based apatite) has especially high adsorbability for protein, that is high adsorbability for cells due to its unique crystal structure.

In the present invention. Ca deficient apatite having a reduced amount of Ca relative to calcium phosphate-based apatite (hereinafter, referred to as “apatite”) is used. The Ca deficient apatite has high adsorbability for protein due to its apatite structure. Accordingly, the cell culture carrier 1 having a surface including its vicinities which is mainly made of such Ca deficient apatite (that is, the coating layer 3 in this embodiment) allows cells to adhere thereto highly efficiently.

Further, according to such cell culture carriers 1, cells that have adhered to the surfaces thereof can more efficiently grow and the grown cells can be easily removed or detached from the carriers 1 as compared to cell culture carriers each having a coating layer 3 which is made of apatite (calcium phosphate-based apatite in which Ca thereof is not substantially deficient). These effects are supposed to result from the following factors.

Namely, cells grow on the surfaces of the cell culture carriers 1 using the proteins as markers. However, in the case where too much Ca are existing on the surfaces of the cell carriers (that is, in the case of apatite), cells are highly likely to directly adhere (that is, nonspecifically adhere) to Ca, thereby disturbing the growth of the cells. On the other hand, the Ca deficient apatite has a reduced amount of Ca relative to the apatite, thereby enabling to reduce the probability that the cells adhere to Ca directly. With this result, the growth of the cells can be accelerated.

Further, generally, bonding force (adherence) of the cells to Ca is strong. However, in the case of the cell culture carrier 1 having the Ca deficient apatite coating, it is possible to decrease the probability that cells directly adhere (nonspecifically adhering) to Ca. For this reason, it is believed that in the cell culture carrier 1 having the Ca deficient apatite coating it is possible to easily remove or detach the grown cells from the cell culture carriers 1.

As described above, according to the cell culture carriers 1 of the present invention, cells are allowed to efficiently adhere thereto and then efficiently grow thereon, while such grown cells can be easily removed or detached therefrom.

In the Ca deficient apatite, the Ca deficiency rate varies slightly depending on the kind of the apatite and is not limited to any specific value. However, it is preferred that the Ca deficient rate is in the range of about 1 to 30 mol %, more preferably in the range of about 5 to 25 mol % and even more preferably in the range of about 8 to 20 mol %. If the Ca deficiency rate is too low, there is a possibility that cells cannot efficiently grow on the surfaces of the cell culture carriers 1, that is, the cell growing rate on the surfaces of the cell culture carriers 1 is decreased. Further, there is also another possibility that the grown cells cannot be efficiently detached from the surfaces of the cell culture carriers 1, that is, removability or detachability of the grown cells is lowered. On the other hand, if the Ca deficiency rate is too high, there is a possibility that cells cannot efficiently adhere to the surfaces of the cell culture carriers 1, that is, adhesion of cells to the surfaces of the cell culture carriers 1 is impaired.

Examples of the Ca deficient apatite include various kind of apatite such as Ca₁₀(PO₄)₆(OH)₂, Ca₁₀(PO₄)₆F₂, Ca₁₀(PO₄)₆Cl or the like in which a part of Ca thereof is deficient. These may be used alone or in combination of two or more of them.

Among them, as the Ca deficient apatite, one containing hydroxyapatite in which a part of Ca is deficient (Ca_{10-x-3y}(PO₄) _{6-2y}(OH)₂₋₂ₓ, where x≦1, y≦3) as a main component is particularly suitable. Hydroxyapatite is used as a biomaterial, and thus cells can highly efficiently adhere thereto, and there is particularly less possibility of damaging the cells. For this reason, the Ca deficient hydroxyapatite can also have such properties.

Further, as the Ca deficient apatite, halogenated apatite such as fluorine apatite (Ca₁₀(PO₄)₆F₂) or chlorine apatite (Ca₁₀(PO₄)₆Cl₂) in which a part of Ca is deficient may be used.

In this regard, the Ca deficient apatite may contain a substance remaining as a resultant of synthesis (a raw material or the like) and/or a secondary reaction product produced in the course of synthesis.

Further, the coating layer 3 may be formed by making the Ca deficient apatite attache to the surface of the base material 2. However, it is preferred that, as shown in FIG. 1, the coating layer 3 is formed from fine particles 31 containing the Ca deficient apatite as a main component (hereinafter, referred to as “particles 31”) which are partially embedded in the surface of the base material 2 including its vicinities. This makes it possible to prevent the coating layer 3 from being peeled off or detached from the surface of the base material 2. Namely, it is possible to provide cell culture carriers 1 having sufficient strength.

Such a coating layer 3 may be either dense or porous.

Furthermore, the average thickness of the coating layers 3 is not limited to any specific value, but is preferably in the range of about 0.1 to 5 μm, and more preferably in the range of about 0.5 to 2 μm. If the average thickness of the coating layers 3 is less than the above lower limit value, there is a case that a part of the surface of the base material 2 is exposed in the cell culture carrier 1. On the other hand, if the average thickness of the coating layers 3 exceeds the above upper limit value, there is a case that it becomes difficult to adjust the density of the cell culture carrier 1.

In terms of enabling a larger number of cells to adhere to and grow on the surfaces of the cell culture carriers 1 described above, it is preferred that substantially the entire surface of each base material 2 is covered with the coating layer 3, similarly to the present embodiment. However, the cell culture carrier 1 may have a structure in which a part of the surf ace of the base material 2 is covered with the coating layer 3, depending on the kind of cell to be cultured by the cell culture carriers 1, the kind of constituent material of the base material 2, or the like (that is, a structure in which a part of the surface of the magnetic particle 2 is exposed through gaps of the coating layer 3).

Hereinafter, a cell culturing method using the above described cell culture carriers 1 according to the present invention will be described.

<1> First, the cell culture carriers 1 are subjected to a sterilization treatment. This makes it possible to decrease the number of microorganisms or molds present on the surfaces of the cell culture carriers 1, or to fully kill the microorganisms or the molds. Therefore, a possibility that the microorganisms or the molds may cause damage to cells is reduced or eliminated, thereby enabling the cells to grow more efficiently.

Examples of the sterilization treatment include a method in which the cell culture carriers 1 are brought into contact with a sterilizing solution, autoclave sterilization, gaseous sterilization, radiation sterilization and the like. Among these methods, the autoclave sterilization is particularly suitable. According to this method, it is possible to more efficiently sterilize a large number of the cell culture carriers 1.

<2> Next, a culture solution containing the cell culture carriers 1 after the completion of the above process <1> and cells (which are allowed to adhere to the cell culture carriers 1) are prepared.

Examples of the cell include an animal cell, a plant cell, a bacterium, a virus, and the like.

The culture solution is appropriately selected depending on the kind of cell to be used, and is not limited to any specific one. Examples of a usable culture solution include Dulbecco's MEM, BME, MCDB-104 medium, and the like.

Further, these culture solutions may contain, for example, serum, serum protein such as albumin, and additives such as various vitamins, amino acid, and salts, if necessary.

Next, the prepared culture solution is agitated. By agitating the culture solution, the cells come into contact with the cell culture carriers 1 and adhere to the surfaces thereof. In such a condition, the cells grow on the surfaces of the cell culture carriers 1 with the lapse of time, namely the cells are cultured. By culturing the cells with agitating the culture solution, it is possible to accelerate the growth efficiency of the cells.

The agitation speed of the culture solution is not limited to any specific value, but is preferably in the range of about 5 to 100 rpm, and more preferably about 10 to 50 rpm. If the agitation speed is too slow, there is a possibility that the cell culture carriers 1 cannot be dispersed in the culture solution uniformly depending on the density and the average particle size of the cell culture carriers 1. In such a case, it is difficult for the cells to sufficiently grow on the surfaces of the cell culture carriers 1. On the other hand, if the agitation speed is too fast, the cell culture carriers 1 are excessively agitated, thereby causing a situation that the cell culture carriers 1 collide with each other, damaging the cells adhering thereto.

Further, the temperature of the culture solution (the temperature for culturing) is appropriately determined depending on the kind of cell to be cultured, and is not limited to any specific value. In usual, the temperature is in the range of about 4 to 40° C., and is preferably in the range of about 25 to 37° C.

<3> Next, the cells that have adhered to and grown on the surfaces of the cell culture carriers 1 are removed or detached from the cell culture carriers 1 and then collected. At first, the cell culture carriers 1 are brought into contact with a cell detachment solution, causing the cells to be removed or detached from the surfaces of the cell culture carriers 1 and then released into the cell detachment solution.

Examples of the cell detachment solution include a trypsin solution, EDTA solution, hypotonic solution, and the like.

The temperature of the cell detachment solution is not limited to any specific value, but is preferably in the range of about 4 to 40° C., and more preferably in the range of about 25 to 37° C.

The time during which the cell culture carriers 1 are being kept in contact with the cell detachment solution is also not limited to any specific value, but is preferably in the range of about 1 to 30 minutes, and more preferably in the range of about 5 to 15 minutes.

Further, while the cell culture carriers 1 are kept in contact with the cell detachment solution, the cell detachment solution may be agitated, given vibration, shaken and the like. In this way, it becomes possible to improve the collection rate of the cells into the cell detachment solution.

At the agitation of the cell detachment solution, the agitation speed is not limited to any specific value, but is preferably in the range of about 5 to 100 rpm, and more preferably in the range of about 10 to 50 rpm.

Next, the cell detachment solution containing the cells is passed through a filter or a column or the like to collect the cells.

The collected cells are utilized in various experiments, researches, production of protein and the like.

For example, such cell culture carriers 1 can be manufactured in accordance with the following processes.

Hereinafter, a method for manufacturing the cell culture carrier 1 shown in FIG. 1 (one example of a method for manufacturing the cell culture carrier according to the present invention) will be described.

The method for manufacturing the cell culture carrier 1 shown in FIG. 1 includes the steps of: obtaining slurry containing apatite and phosphoric acid; drying the slurry to obtain powder; sintering the dried powder to obtain sintered powder; and coating the surface of a base material with the sintered powder. Hereinafter, each of the steps will be described in detail in this order.

[A] Step for Obtaining Slurry (First Step)

First, slurry containing apatite and phosphoric acid is prepared.

This is carried out by, for example, a method in which phosphoric acid is added to an apatite dispersion liquid (slurry), a method in which apatite is added to a phosphoric acid solution, a method in which an apatite dispersion liquid and a phosphoric acid solution are mixed, a method in which apatite and phosphoric acid are mixed and then liquid (dispersion medium) is added thereto.

Further, apatite can be synthesized by a well-known method such as a wet synthetic method, dry synthetic method, or the like.

The mixing ratio between apatite and phosphoric acid is not limited to any specific value, but preferably in the range of about 6:1 to 1:1 in a molar ratio, more preferably in the range of about 6:3 to 6:5. If the amount (added amount) of phosphoric acid is too small, the Ca deficiency rate in the sintered powder to be obtained in the following step [C], which will be described later, may become lower, thereby causing a situation that cells cannot sufficiently grow on the manufactured cell culture carriers 1 and cell cannot be detached from the manufactured cell culture carriers 1. On the other hand, if the amount of phosphoric acid is too large, the Ca deficiency rate in the sintered power to be obtained may become unnecessarily high, thereby causing a situation that cells cannot sufficiently adhere to the manufactured cell culture carriers 1.

[B] Step for Obtaining Powder (Second Step)

Next, the slurry that has been obtained in the above step [A] is dried to obtain powder.

Examples of a method for drying the slurry include heated-air drying, freeze drying, vacuum drying, spray drying and the like.

When applying heat to the slurry for drying, the heating temperature is preferably in the range of about 40 to 300° C., and more preferably in the range of about 80 to 250° C.

Further, for example, in the method such as spray drying in which the dried slurry becomes a powder (grain) form, the dried slurry in the form of powder can be directly sent to the next step [C]. On the other hand, in the method in which the dried slurry becomes a block form, such a block is milled before the next step [C].

The average particle size of the powder is not limited to any specific value, but is preferably equal to or less than 30 μm. In particular, the average particle size of the powder is preferably in the range of about 1 to 50% of the diameter of the base material 2, and more preferably in the range of about 10 to 25 t. According to this, it is possible to more easily form the coating layer 3 having a desired thickness.

[C] Step for Sintering Dried Powder (Third Step)

Next, the powder that has been obtained in the above step [B] is sintered to obtain sintered powder.

In the present invention, the sintering of the dried powder is carried out at a temperature that can keep its apatite structure. In this way, Ca deficient apatite is produced by the reaction between the apatite and the phosphoric acid in the powder, so that the sintered powder that is mainly constituted of the Ca deficient apatite can be obtained.

In this regard, it is to be noted that if the powder is sintered at a temperature higher than the temperature at which the apatite structure can be maintained, a chemical reaction indicated by the following chemical formula occurs.
3Ca₁₀(PO₄)₆(OH)₂+2H₃(PO₄)→10Ca₃(PO₄)₂+6H₂O (I)

Namely, apatite (Ca₁₀(PO₄)₆(OH)₂) is changed into tricalcium phosphate (Ca₃(PO₄)₂:TCP).

As a result, in the sintered powder obtained, the content of the Ca deficient apatite is reduced, and thus cells cannot efficiently adhere to and grow on the surfaces of the cell culture carriers 1 finally obtained.

Further, although the above chemical formula (I) was shown with regard to the case where hydroxyapatite is used, the same result can be obtained in the case where the other kind of apatite is used.

The sintering temperature varies slightly depending on the kind of apatite and is not limited to any specific value, but is preferably equal to or less than 1000° C., and more preferably in the range of about 400 to 750° C. The sinterenig of the powder in the above temperature range makes it possible to more reliably produce the Ca deficient apatite.

Further, the sintering time is preferably in the range of about 0.1 to 10 hours, and more preferably in the range of about 2 to 4 hours. If the sintering time is too short, there is a possibility that it becomes difficult to produce a sufficient amount of the Ca deficient apatite. On the other hand, even if the sintering time is made longer than the above indicated upper limit, further progress of the chemical reaction can not be expected, and thus it is not desirable because of merely making the total manufacturing time longer.

Further, the sintering atmosphere is not limited to any specific one. For example, oxygen-containing atmosphere such as atmospheric air or pure oxygen atmosphere, argon-containing atmosphere, nitrogen-containing atmosphere and the like can be employed, but atmospheric air is particularly preferable. This makes it is possible to efficiently produce the Ca deficient apatite as well as to save manufacturing costs.

[D] Step for Coating Base Material (Fourth Step)

Next, the surface of the base material 2 is coated with the sintered powder that has been obtained in the above step [C] to form the coating layer 3.

When manufacturing the cell culture carrier 1 as shown in FIG. 1, namely when forming the coating layer 3 so that particles 31 are partially embedded in a surface area of the base material 2, the coating layer 3 can be formed by colliding the sintered powder with the surface of the base material 2. According to this method, it is possible to easily and reliably form the coating layer 3 having uniform thickness.

The collision between the base material 2 and the sintered powder can be carried out in a dry condition using a commercially available machine called “MECHANOFUSION” (Trademark of Hosokawa Micron Co., Ltd.), for example. In such a case, the collision is carried out under the condition that the mixing ratio between the base material 2 and the sintered powder is about 400:1 to 10:1 in a weight ratio, and the temperature inside the machine is equal to or less than the softening temperature of the resin material which is used as a main material of the base material 2 (normally, equal to or lower than 80° C.), for example. In this regard, it is to be noted that a method for forming the coating layer 3 is not limited the above-mentioned method.

The cell culture carriers 1 are obtained through the steps described above.

In this regard, it should be also noted that the above-mentioned sintered powder can be used as cell culture carriers 1 as they are. In such a case, the step [D] is omitted.

### Second Embodiment

Next, the cell culture carriers according to the second embodiment of the present invention will be described.

Hereinafter, a description will be made by focusing the difference between the cell culture carriers according to the first embodiment and the second embodiment, and therefore a description of overlapping points will be omitted.

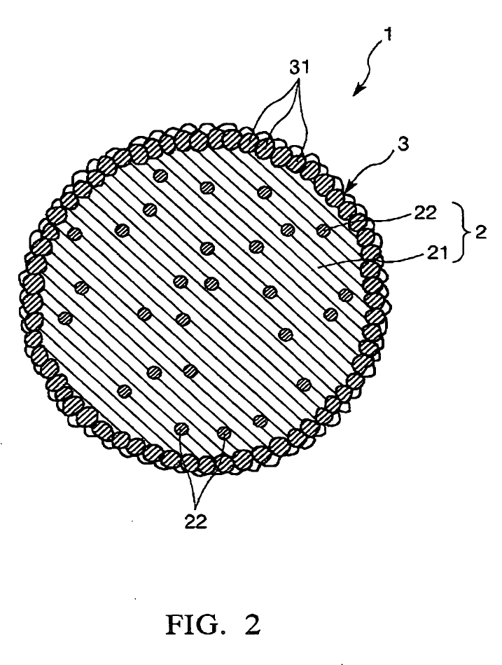

FIG. 2 is a cross-sectional view of a cell culture carrier according to the second embodiment of the present invention.

The cell culture carrier 1 shown in FIG. 2 and the cell culture carrier 1 according to the first embodiment have the same structure excepting the base material 2.

The base material 2 shown in FIG. 2 contains magnetic materials 22 within the resin material 21 which is a main component thereof, thus the cell culture carrier 1 has magnetic property as a whole.

Because of the above structure, the cell culture carriers 1 can be moved in a culture solution when a magnetic field is applied thereto so that it is possible to agitate the culture solution by the movement of the cell culture carriers 1. Therefore, it is possible to prevent mechanical shock from being added to the cell culture carriers 1, which would be caused in the conventional culture method using a spinner flask due to collision between a fin (stirring bar) and cell culture carriers. This makes it possible to prevent the cells adhering to the cell culture carriers 1 from being removed or detached from the surfaces thereof and also to prevent the cells from being damaged.

Further, as compared to an agitation using a stirring bar, the agitation of the culture solution by the cell culture carriers 1 described above allows the culture solution to be agitated more uniformly and gently (mildly). Therefore, the cells easily adhere to the surfaces of the cell culture carriers 1, and nutrition is equally supplied to the cells adhering on the surfaces of the carriers. Therefore, these cell culture carriers 1 serve as good scaffolds for allowing the cells to grow.

The entire base material 2 may be formed of a magnetic material, but it is preferred that the base material 2 is formed of a composite particle which is obtained by compounding a resin material 21 and a magnetic material 22 as the present embodiment. According to this structure, it is possible to adjust the density (specific gravity) of the base material 2 (that is, each cell culture carrier 1) easily by setting a compounding ratio (mixing ratio) of the resin material 21 and the magnetic material 22 appropriately. Further, there is another advantage in that the shape and size (average particle size or the like) of the cell culture carriers 1 can be easily adjusted.

As for the structure of the composite particle (magnetic particle), it is preferred that, as shown in FIG. 2, a magnetic material (magnetic powder) 22 is dispersed in a resin material 21. Such a base material 2 can be relatively easily manufactured by molding or granulating a resin material 21 in a molten state to which the magnetic material 22 has been mixed. In this regard, it is to be noted that in this composite particle, the magnetic material 22 may be dispersed only in a portion of the resin material 21 which is located in the vicinity of the surface thereof.

Examples of the magnetic material 22 include a ferromagnetic alloy containing iron oxide, Fe, Ni, Co. or the like as a main component thereof, ferrite, barium ferrite, strontium ferrite, and the like. These magnetic materials may be used alone or in combination of two or more.

According to the cell culture carriers of the second embodiment described above, it is possible to obtain the same operation and effect as those of the cell culture carrier according to the first embodiment.

Further, such a cell culture carrier 1 of the second embodiment can be manufactured in the same manner as in the first embodiment.

Further, in each embodiment described above, the surface including its vicinities of the cell culture carrier 1 (namely, the coating layer 3) is mainly made of the calcium phosphate-based apatite in which a part of Ca is deficient. However, the surface including its vicinities of the cell culture carrier 1 of the present invention may be mainly made of apatite in which a part of bivalent element thereof is deficient.

Examples of such apatite include, Mg₁₀(PO₄)₆(OH)₂. Cd₁₀(PO₄)₆(OH)₂, Sr₁₀(PO₄)₆(OH)₂, Pb₁₀(PO₄)₆(OH)₂, Ba₁₀(PO₄)₆(OH)₂, Mg₁₀(SO₄)₆(OH)₂ and the like.

Although the cell culture carriers and the method for manufacturing the cell culture carriers according to the present invention have been described in the above, the present invention is not limited thereto.

For example, the shape of each cell culture carrier is not limited to a particle shape and it may be formed into various shapes such as a block shape, pellet shape, sheet shape or the like.

Further, the entire of the cell culture carrier may be formed of a calcium phosphate-based compound.

Furthermore, the method for manufacturing the cell culture carriers of the present invention may further include one or more additional steps for arbitral purpose in addition to the above-described steps, if necessary.

### EXAMPLES

Next, actual examples of the present invention will be described.

1. Study of Sintering Temperature for Powder

1-1. Production of Sintered Powder

### Experimental Example 1

<1> First, hydroxyapatite was synthesized by a wet synthetic method.

<2> Next, 10 wt % of a hydraxyapatite dispersion water was prepared.

<3> Next, 15 g of 10 wt % of phosphoric acid solution was added to 1 liter of the hydroxyapatite dispersion water to obtain slurry.

<4> Next, the slurry was dried at the temperature of 200° C. for 2 hours and then ground in a mortar to obtain powder having an average particle size of 20 μm.

<5> Next, the obtained powder was sintered at the temperature of 700° C. for 2 hours to obtain sintered powder.

### Experimental Example 2

Cell culture carriers were obtained in the same manner as in Experimental Example 1 except that the sintering temperature for the powder was changed to 1100° C. in the above step <5>.

### Experimental Example 3

Cell culture carriers were obtained in the same manner as in Experimental Example 1 except that the sintering temperature for the powder was changed to 1200° C. in the above step <5>.

### Experimental Example 4

Cell culture carriers were obtained in the same manner as in Experimental Example 1 except that the amount of phosphoric acid solution added to the hydroxyapatite dispersion water was changed to 45 g in the above step <3>.

### Experimental Example 5

Cell culture carriers were obtained in the same manner as in Experimental Example 4 except that the sintering temperature for the powder was changed to 1100° C. in the above step <5>.

Cell culture carriers were obtained in the same manner as in Experimental Example 4 except that the sintering temperature for the powder was changed to 1200° C. in the above step <5>.

1-2. Crystal Structure Analysis for Sintered Powder

A crystal structure analysis for the sintered powder was carried out by an X-ray diffraction method for each sintered powder obtained in respective experimental examples.

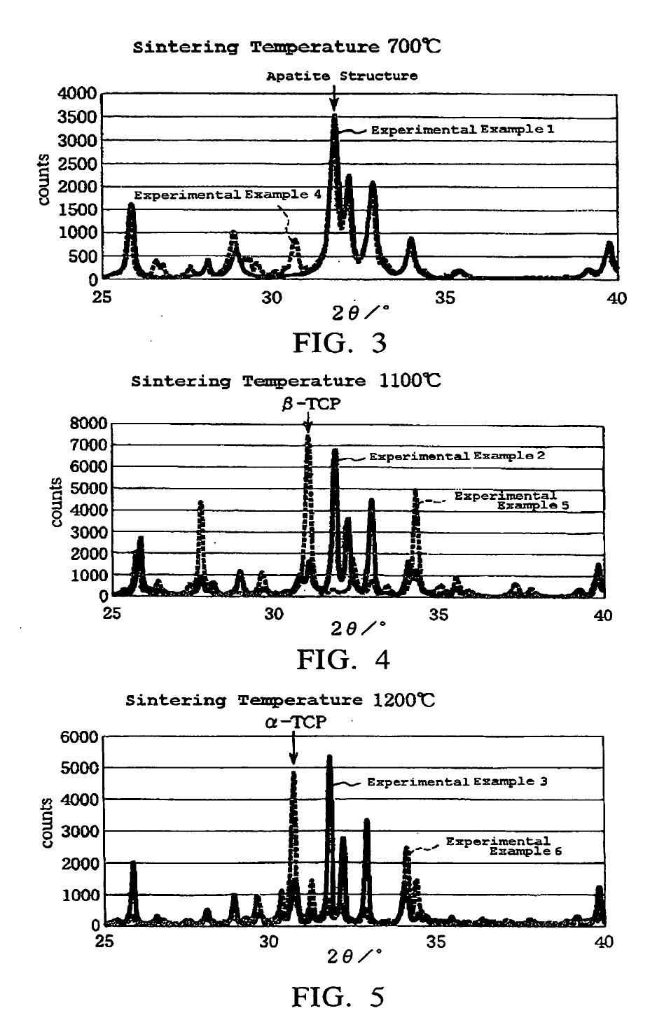

The results thereof are shown in FIG. 3 to FIG. 5.

As shown in FIG. 3, when the sintering temperature was set at 700° C. (Experimental Examples 1 and 4), a large peak derived from apatite was observed while a peak derived from TCP (tricalcium phosphate) was not recognized.

On the other hand, when the sintering temperature was set at 1100° C. (Experimental Examples 2 and 5), as shown in FIG. 4, a peak derived from β-TCP was observed. Further, when the sintering temperature was set at 1200° C. (Experimental Examples 3 and 6), a peak derived from A-TCP was observed.

In particular, in the sintered powder of each of Experimental Examples 5 and 6 in which the amount of the addition of the phosphoric acid was increased with respect to the hydroxyapatite, the largest peaks derived from β-TCP and α-TCP were respectively observed while a peak derived from apatite was hardly recognized.

By the results of these analyses, it has been confirmed that an appropriate sintering temperature is needed to obtain the Ca deficient hydroxyapatite.

2. Evaluation of Cell Culture Carrier

2-1. Production of Cell Culture Carrier

### Example 1

At first, sintered powder was obtained in the same manner as in the Experimental Example 1. Further, the Ca deficiency rate was measured by the X-ray diffraction method, and it was 3 mol %. (Note that in the following examples, the same method was used.)

In particular, the measurement of the Ca deficiency rate was carried out in accordance with the following manners.

First, a main peak intensity regarding each of a simple substance of hydroxyapatite and a simple substance of tricalcium phosphate (β-TCP) both sintered at the temperature of 1100° C. was respectively measured by the X-ray diffraction measurement to obtain a reference value.

As a result, the number of counts of the simple substance of hydroxyapatite was 5,000 counts and the number of counts of the simple substance of single β-TCP was 3,500 counts.

Next, the Ca deficient hydroxyapatite (sintered powder) that has been obtained in Experimental Example 1 was sintered at the temperature of 1100° C. and then subjected to the X-ray diffraction measurement.

As a result, X-ray diffraction pattern including both the hydroxyapatite and the β-TCP was obtained.

The number of counts of the hydroxyapatite at the main peak was 3,500 counts and that of the β-TCP was 1,050 counts.

In this regard, it is to be noted that the sintering at the temperature of 1100° C. was carried out for the purpose of improving crystallinity of each sintered powder and causing phase transition from the Ca deficient hydroxyapatite to the β-TCP.

Next, the Ca deficiency rate was calculated based on the reference value obtained in the above and from the number of counts obtained when the Ca deficient hydroxyapatite was measured in the above.

The structural formula (composition formula) of the ca deficient apatite is the sum of 3500/5000(Ca₁₀(PO₄)₆(OH)₂)═Ca_{9.7}(PO)_{4.2}(OH)_{1.4} and 1050/3500(Ca₉(PO₄)₆)═Ca_{2.7}(PO₄)_{1.8}, namely Ca_{9.7}(PO₄)₆(OH)_{1.4}.

Normally, hydroxyapatite contains 10 mol of calcium. However, the Ca deficient hydroxyapatite (sintered powder) contains only 9.7 mol of calcium, which indicates that the Ca deficiency rate is 3 mol %.

In this regard, it is to be noted that although β-TCP is normally indicated by the composition formula of Ca₃(PO₄)₂, it is indicated by Ca₉(PO₄)₆ In the above for calculation convenience.

Further, nylon particles (base materials) having an average particle size of 150 μm and a density of 1.02 g/cm³ were prepared.

Next, 3 g of the sintered powder and 300 g of the nylon particles were put into a “MECHANOFUSION SYSTEM AMS-LAB” (manufactured by Hosokawa Micron Co. Ltd.), and the system was operated at 2,650 rpm at 45° C. for 70 minutes. In this way, cell culture carriers 1 as shown in FIG. 1 were obtained.

The thus obtained cell culture carriers 1 had an average particle size of 151 μm (the average thickness of the coating layer was 1 μm) and a density of 1.03 g/cm³.

### Example 2

Sintered powder was obtained in the same manner as in Experimental Example 1 except that the amount of phosphoric acid solution to be added to the water dispersion was changed to 30 g. In this example, the Ca deficiency rate was 5 mol %. Then, cell culture carriers were obtained using this sintered powder in the same manner as in Example 1.

### Example 3

First, sintered powder was obtained in the same manner as in Experimental Example 4. In this example, the Ca deficiency rate was 11 mol %. Then, cell culture carriers were obtained with this sintered powder in the same manner as in Example 1.

### Example 4

Sintered powder was obtained in the same manner as in Experimental Example 1 except that the amount of phosphoric acid solution to be added to the water dispersion was changed to 50 g. In this example, the Ca deficiency rate was 15 mol %. Then, cell culture carriers-were obtained using this sintered powder in the same manner as in Example 1.

### Example 5

Sintered powder was obtained in the same manner as in Experimental Example 1 except that the amount of phosphoric acid solution to be added to the water dispersion was changed to 55 g. In this example, the Ca deficiency rate was 19 mol %. Then, cell culture carriers were obtained using this sintered powder in the same manner as in Example 1.

### Example 6

Sintered powder was obtained in the same manner as in Experimental Example 1 except that the amount of phosphoric acid solution to be added to the water dispersion was changed to 60 g. In this example, the Ca deficiency rate was 23 mol %. Then, cell culture carriers were obtained using this sintered powder in the same manner as in Example 1.

### Example 7

Sintered powder was obtained in the same manner as in Experimental Example 1 except that the amount of phosphoric acid solution to be added to water dispersion was changed to 65 g. In this example, the Ca deficiency rate was 29 mol %. Then, cell culture carriers were obtained using this sintered powder in the same manner as in Example 1.

### Example 8

Sintered powder was obtained in the same manner as in Experimental Example 4 except that fluorine apatite was used instead of hydroxyapatite. In this example, the Ca deficiency rate was 12 mol %. Then, cell culture carriers were obtained using this sintered powder in the same manner as in Example 1.

### Example 9

Sintered powder was obtained in the same manner as in Experimental Example 4 except that chlorine apatite was used instead of hydroxyapatite. In this example, the Ca deficiency rate was 10 mol %. Then, cell culture carriers were obtained using this sintered powder in the same manner as in Example 1.

### Example 10

The cell culture carriers as shown in FIG. 2 were obtained in the same manner as in Example 1 except that ferrite composite nylon particles were used as a base material instead of nylon particles.

The thus obtained cell culture carriers had an average particle size of 151 μm (the average thickness of coating layer was 1.0 μm), and a density of 1.23 g/cm³.

### Comparative Example 1

First, sintered powder was obtained in the same manner as in Experimental Example 1 except that the amount of phosphoric acid solution to be added to the water dispersion was changed to 0 g. In this example, the Ca deficiency rate was 0 mol %. Then, cell culture carriers were obtained using this sintered powder in the same manner as in Example 1.

### Comparative Example 2

First, sintered powder was obtained in the same manner as in Experimental Example 1 except that fluorine apatite was used instead of hydroxyapatite and the amount of phosphoric acid solution to be added to the water dispersion was changed to 0 g. In this example, the Ca deficiency rate was 0 mol %. Then, cell culture carriers were obtained using this sintered powder in the same manner as in Example 1.

### Comparative Example 3

First, sintered powder was obtained in the same manner as in Experimental Example 1 except that chlorine apatite was used instead of hydroxyapatite and the amount of phosphoric acid solution to be added to the water dispersion was changed to 0 g. In this example, the Ca deficiency rate was 0 mol %. Then, cell culture carriers were obtained using this sintered powder in the same manner as in Example 1.

2-2. Cell Culture

<I> Cell culture was carried out using the cell culture carriers obtained In each of Examples 1 to 9 and Comparative Examples 1 to 3 in accordance with the following manners.

First, each of the cell culture carriers obtained in each of Examples and each of Comparative Examples were subject to autoclave sterilization.

Next, 1 g of the cell culture carriers and a suspension containing 2×10⁵ vero cells derived from an African green monkey kidney per milliliter (/mL) were added to 100 mL of MEM medium (culture solution).

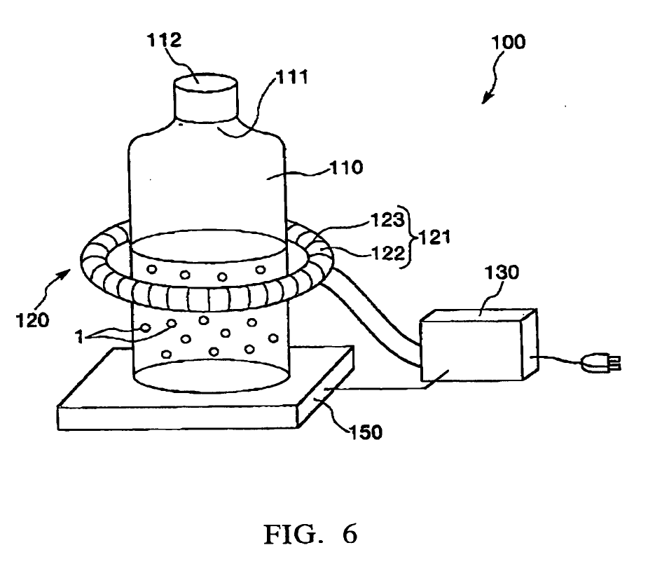

This culture solution was put in a spinner flask, and the cells were cultured under the conditions that a rotational speed was 30 rpm, a temperature of the culture solution was 37° C., and a cultivation period was 3 days.

<II> Cell culture was carried out by use of a cell culture apparatus shown in FIG. 6 in accordance with the following manners using the cell culture carriers obtained in Example 10.

Now, the structure of the cell culture apparatus 100 will be described.

A cell culture apparatus 100 shown in FIG. 6 has a culture vessel 110, a magnetic field generator 120, a controller 130, and a heating device 150. When the controller 130 is connected to a power source, electric power necessary to actuate each of the components of the cell culture apparatus 100 is supplied.

The culture vessel 110 is a component for receiving the culture solution, and has an opening 111, through which the culture solution is fed and discharged, at the upper portion thereof. The opening 111 is closed with a plug 112 to maintain an airtight condition within the culture vessel 110.

The magnetic field generator 120 is a component for generating a magnetic field to move the cell culture carriers 1 in the culture solution and has an electromagnet 121 that is provided so as to surround the periphery of the culture vessel 110.

The electromagnet 121 is comprised of a toroidal metallic core material 122 and a conductor 123 spirally wound around the periphery of the core material 122. The passage of electric current through the conductor 123 generates a magnetic field in the vicinity of the conductor 123.

When a magnetic field is generated by the magnetic field generator 120, the cell culture carriers 1 are attracted to the side of the magnetic field generator 120 so that the cell culture carriers 1 rise in the culture solution. In such a state, when the generation of the magnetic field is stopped, the cell culture carriers 1 attracted to the side of the magnetic field generator 120 settle down under their own weight. By repeating such a vertical movement of the cell culture carriers 1, a turbulent flow is generated in the culture solution so that the culture solution is uniformly and gently agitated.

Further, the heating device 150 is electrically connected to the controller 130 to heat the culture solution under the control of the controller 130.

Next, a method for culturing cells will be described.

First, the cell culture carriers of Example 10 were subject to autoclave sterilization.

Next, 1 g of the cell culture carriers and a suspension containing 2×10⁵ vero cells derived from an African green monkey kidney per milliliter (/mL) were added to 100 mL of MEM medium (culture solution).

This culture solution was put in the culture vessel 110 and heated at the temperature of 37° C.

In such a state, cell culture has been carried out for three days by setting the magnetic field generator 120 so that the magnetic field was intermittently generated at regular intervals. Further, strength of the magnetic field to be generated was set to 0.5 Wb/m².

Such a cell culture operation was carried out 10 times in each of Examples and each of Comparative Examples.

In 5 out of 10 times, the cell culture carriers that had been cultured for three days were taken out from the culture solution and then the metabolic activity of the cells was measured through Alamar blue reduction method.

On the other hand, in the remaining 5 times, the cell culture carriers that had been cultured for three days were taken out from the culture solution and then put in 2 mL of trypsin solution (collecting solution) for 5 minutes to detach the cells from the cell culture carriers 1.

Next, the cell culture carriers were taken out from the trypsin solution and then the metabolic activity of the cells was measured through the Alamar blue reduction method.

These results are shown in Table 1 with the Ca deficiency rate.

In this regard, it is to be noted that each cellular metabolic activity value shown in Table 1 is a relative value when the cellualr metabolic activity value before the cells were removed or detached from the cell culture carriers in Comparative Example 1 is defined as 100.

Further, each value is an average value of 5 times.

Table 1

TABLE 1

| | | Ca | Metabolic activity of Cell | |
|---|---|---|---|---|
| | | deficiency | Before | After |
| | | rate | Removing | Removing |
| | Kind of Apatite | [mol %] | Cell | Cell |
| Ex. 1 | Hydroxyapatite | 3 | 115 | 30 |
| Ex. 2 | Hydroxyapatite | 5 | 130 | 27 |
| Ex. 3 | Hydroxyapatite | 11 | 154 | 25 |
| Ex. 4 | Hydroxyapatite | 15 | 160 | 24 |
| Ex. 5 | Hydroxyapatite | 19 | 151 | 23 |
| Ex. 6 | Hydzoxyapatite | 23 | 131 | 26 |
| Ex. 7 | Hydroxyapatite | 29 | 119 | 29 |
| Ex. 8 | Fluorineapatiete | 12 | 155 | 35 |
| Ex. 9 | Chlorineapatite | 10 | 148 | 33 |
| Ex. 10 | Hydroxyapatite | 3 | 132 | 32 |
| Com. Ex. 1 | Hydroxyapatite | 0 | 100 | 53 |
| Com. Ex. 2 | Fluorineapatiete | 0 | 95 | 45 |
| Com. Ex. 3 | Chlorineapatite | 0 | 88 | 43 |

As shown in Table 1, in each of Examples, the metabolic activity value of the cell culture carriers at the time after three days had been passed from the start of the cell culture (that is, before the cells were removed or detached from the cell culture carriers) was apparently higher than the value shown in each of Comparative Examples. Further, the results show a tendency that in each of Examples the metabolic activity of the cell culture carriers at the time after three days had been past from the start of the cell culture (that is, before the cells were removed or detached from the cell culture carriers) became higher by appropriately setting the Ca deficiency rate.

Further, in each of Examples, the metabolic activity value of the cell culture carriers was apparently decreased by removing or detaching the cells from the cell culture carriers. On the other hand, in each of Comparative Examples, the metabolic activity value did not show a big change even after the cells were removed or detached from the cell culture carriers.

From these results, it has been found that the cell culture carriers according to the present invention allow cells to efficiently adhere thereto and satisfactorily grow thereon in addition to the fact that the grown cells can be easily removed or detached from the cell culture carriers.

Further, it has also been found that according to the cell culture carriers of the present invention it is possible for cells to grow faster (more efficiently) and it is also possible to improve removability or detachability of the grown cells from the cell culture carriers by appropriately setting the Ca deficiency rate.

Finally, it is to be understood that many changes and additions may be made to the embodiments described above without departing from the scope and spirit of the invention as defined in the following claims.

Further, it is also to be understood that the present disclosure relates to subject matter contained in Japanese Patent Application No. 2004-220870 (filed on Jul. 28, 2004) which is expressly incorporated herein by reference in its entirety.

What is claimed is:

1. Cell culture carriers each having a surface to which cells are allowed to adhere so that the adhered cells grow on the surfaces thereof, wherein each of the cell culture carriers has at least a surface thereof including its vicinities mainly made of calcium phosphate-based apatite in which a part of Ca is deficient.

2. The cell culture carriers as claimed in claim 1, wherein the Ca deficiency rate of the calcium phosphate-based apatite in which a part of Ca is deficient is 1 to 30 mol %.

3. The cell culture carriers as claimed in claim 1, wherein the density of each of the cell culture carriers is in the range of 1.01 to 1.5 g/cm³.

4. The cell culture carriers as claimed in claim 1, wherein each of the cell culture carriers has a particle shape.

5. The cell culture carriers as claimed in claim 4, wherein an average particle size of the cell culture carriers is in the rage of 10 to 2000 μm.

6. The cell culture carriers as claimed in claim 1, wherein the calcium phosphate-based apatite in which a part of Ca is deficient is mainly made of hydroxyapatite in which a part of Ca is deficient.

7. The cell culture carriers as claimed in claim 1, wherein each of the cell culture carriers comprises a base material having a surface and a coating layer formed so as to coat the surface of the base material, the coating layer being mainly made of calcium phosphate-based apatite in which a part of Ca is deficient.

8. The cell culture carriers as claimed in claim 7, wherein the base material is mainly made of a resin material.

9. The cell culture carriers as claimed in claim 8, wherein the resin material is mainly composed of at least one of polyamide and epoxy resin.

10. The cell culture carriers as claimed in claim 7, wherein the base material contains a magnetic material.

11. Cell culture carriers each having a surface to which cells are allowed to adhere so that the adhered cells grow on the surfaces thereof, wherein each of the cell culture carriers has at least a surface thereof and its vicinities mainly made of apatite in which a part of bivalent element thereof is deficient.

12. A method for manufacturing the cell culture carriers as defined in claim 1, comprising:
a first step for obtaining slurry containing calcium phosphate-based apatite and phosphoric acid;
a second step for drying the slurry to obtain powder; and
a third step for sintering the powder to obtain sintered powder which is mainly formed of the calcium phosphate-based apatite in which a part of Ca is deficient by reacting the calcium phosphate-based apatite and the phosphoric acid in the powder with keeping the apatite structure to thereby produce the calcium phosphate-based apatite in which a part of Ca is deficient.

13. A method for manufacturing cell culture carriers, the method is used for manufacturing the cell culture carriers as defined in claim 7, comprising:
a first step for obtaining slurry containing calcium phosphate-based apatite and phosphoric acid;
a second step for drying the slurry to obtain powder;
a third step for sintering the powder to obtain sintered powder which is mainly formed of the calcium phosphate-based apatite in which a part of Ca is deficient by reacting the calcium phosphate-based apatite and the phosphoric acid in the powder with keeping the apatite structure to thereby produce the calcium phosphate-based apatite in which a part of Ca is deficienta; and
a fourth step for coating a base material with the sintered powder.

14. A method for manufacturing cell culture carriers as claimed in claim 12, wherein a mixing ratio of the calcium phosphate-based apatite and the phosphoric acid is in the range of 6:1 to 1:1 in a molar ratio in the first step.

15. The method for manufacturing cell culture carriers as claimed in claim 12, wherein the sintering temperature in the third step is equal to or less than 1000° C.

16. The method for manufacturing cell culture carriers as claimed in claim 12, wherein the sintering time in the third step is of 0.1 to 10 hours.

17. The method for manufacturing cell culture carriers as claimed in claim 12, wherein the sintering in the third step is carried out in atmospheric air.

18. A method for culturing cells, wherein the cell culture is carried out by using the cell culture carriers as defined in claim 1.

19. A method for culturing cells, wherein in a culture solution containing the cell culture carriers as defined in claim 1 and cells, the cells are brought into contact with the cell culture carriers so that the cells adhere to the surfaces of the cell culture carriers and grow thereon.

20. The method for culturing cells as claimed in claim 18, wherein the cells that have grown on the cell culture carriers are removed therefrom.
